(19) United States
(12) Patent Application Publication
(10) Pub. No.: US 2014/0114580 A1
(43) Pub. Date: Apr. 24, 2014

(54) COMPUTERIZED METHOD AND DEVICE FOR ANALYZING PHYSIOLOGICAL SIGNAL

(71) Applicant: INDUSTRIAL TECHNOLOGY RESEARCH INSTITUTE, (US)

(72) Inventors: Ming-Yen Chen, Tainan City (TW); Chuan-Wei Ting, Kaohsiung City (TW); Ching-Yao Wang, Tainan City (TW)

(73) Assignee: INDUSTRIAL TECHNOLOGY RESEARCH INSTITUTE, Hsinchu (TW)

(21) Appl. No.: 13/870,079

(22) Filed: Apr. 25, 2013

(30) Foreign Application Priority Data
Oct. 24, 2012 (TW) 101139218

(51) Int. Cl. A61B 5/00; A61B 5/029

(52) U.S. Cl. CPC A61B 5/7235; A61B 5/029
USPC 702/19

(57) ABSTRACT

A computerized method and device for analyzing a physiological signal are provided. The computerized method for analyzing the physiological signal includes the following steps. A pulse waveform is measured by a measuring unit, wherein the pulse waveform represents a blood volume of a blood vessel over time. A plurality of rising segments of the plus waveform is analyzed by a processing unit. The maximum change rate point at each rising segment is analyzed by the processing unit. A pulse interval time sequence is obtained according to the maximum change rate points.

This application claims the benefit of Taiwan application Serial No. 101139218, filed Oct. 24, 2012, the disclosure of which is incorporated by reference herein in its entirety.

### TECHNICAL FIELD

The disclosure relates in general to a computerized method and device, and more particularly to a computerized method and a computerized device for analyzing a physiological signal.

### BACKGROUND

In recent years, due to the growth of aging population, many developed countries' expenditures in healthcare expand and are eager to work out a solution to reduce the expenditure in medical care. Due to the uneven distribution of medical care and the huge gap between urban and rural areas, many developing countries are also very concerned about the distribution of the resources of medical care. In view of such trend, global medical care system starts to make adjustment by reducing the expenditure in disease treatment and increasing the expenditure in disease prevention and health promotion. The providers of medical care are extended to health checkup centers, communities, schools, enterprises or even personal studios from institutes of professional medical care. The focus moves to preventive health from disease treatment. The distribution is directed towards decentralized healthcare from centralized healthcare. Furthermore, through the integration of information technology and personal portable devices, healthcare is electronized and mobilized.

As healthcare is directed decentralization, electronization and mobilization, non-invasive pulse signal measuring method and technology are provided. For example, through the pulse waveform obtained by the pulse measuring technology, parameters reflecting the state of cardio vessel health, such as the blood vessel stiffness index (SI) and the blood vessel reflection index (RI), can be detected. The number and interval of the testee's heart beats can be detected through the peaks of the pulse waveform (percussion wave). The time sequence formed by the peak-peak interval (PPI) of the pulse waveform can be regarded as an RRI sequence obtained by electrocardiography (ECG). Then, more parameters reflecting the psychological and physiological states of the testee's health can be promptly obtained through the analysis of heart rate variability (HRV). Various decentralized, electronized and mobilized measuring methods are provided to replace the complicated precision apparatuses used in institutes of medical care, so that quality medical care becomes more popular and promptly accessible to the public.

The pulse waveform measuring methods must consider the influence of the testing environment, so that the precision of measurement can be increased and practical effect can be achieved.

### SUMMARY

The disclosure is directed to a computerized method and a computerized device for analyzing a physiological signal which increase the precision of measurement through the analysis of maximum change rate point.

According to one embodiment, a computerized method for analyzing a physiological signal is provided. The computerized method for analyzing the physiological signal includes the following steps. A pulse waveform is measured by a measuring unit, wherein the pulse waveform represents a blood volume of a blood vessel over time. A plurality of rising segments of the plus waveform is analyzed by a processing unit. The maximum change rate point at each rising segment is analyzed by the processing unit. A pulse interval time sequence is obtained according to the maximum change rate points.

According to another embodiment, a computerized device for analyzing a physiological signal is provided. The computerized device for analyzing the physiological signal includes a measuring unit, a processing unit and a storage unit. The measuring unit measures a pulse waveform. The pulse waveform represents a blood volume of a blood vessel over time. The processing unit is used for analyzing a plurality of rising segments of the plus waveform, and analyzes a maximum change rate point at each rising segment. The storage unit stores the maximum change rate points. The processing unit further obtains a pulse interval time sequence according to the maximum change rate points.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 shows a block diagram of a computerized device for analyzing a physiological signal;

FIG. 2 shows a schematic diagram of a smart phone;

FIG. 3 shows a schematic diagram of a pulse waveform;

FIG. 4 shows a schematic diagram of a blood vessel inside a testing portion of a human body;

FIG. 5 shows a flowchart of a computerized method for analyzing a physiological signal;

FIG. 6 shows a schematic diagram of a pulse waveform measured by a measuring unit;

FIG. 7 shows a schematic diagram of a first derivative function curve of the pulse waveform of FIG. 6;

FIG. 8 shows a schematic diagram of another pulse waveform measured by a measuring unit;

FIG. 9 a schematic diagram of a first derivative function curve of the pulse waveform of FIG. 8;

FIG. 10 shows a comparison of three types of HRV indexes; and

FIG. 11 shows a schematic diagram of a light source, a photo-electro converter and a server.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

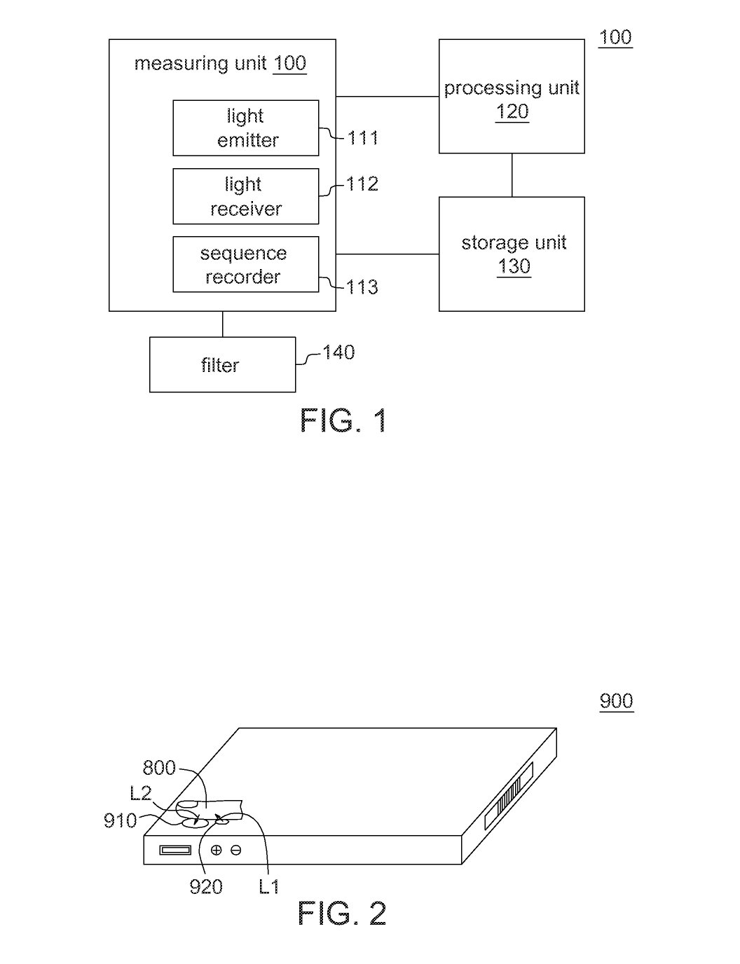

Referring to FIG. 1, a block diagram of a computerized device 100 for analyzing a physiological signal is shown. The computerized device 100 for analyzing the physiological signal includes a measuring unit 110, a processing unit 120 and a storage unit 130. The measuring unit 110 measures various types of physiological information, and can be realized by such as an airbags blood pressure measurer, a photodiode, or a camera lens. The processing unit 120 executes various processing procedures, and can be realized by such as a processing chip, a firmware circuit, or a computer readable recording medium storing a plurality of programming codes. The storage unit 130 stores various types of information, and can be realized by such as a memory, a memory card or a hard disc.

Referring to FIG. 2, a schematic diagram of a smart phone 900 is shown. The computerized device 100 for analyzing the physiological signal can be realized by a multi-function composite electronic device. For example, the computerized device 100 for analyzing the physiological signal can be realized by a smart phone 900. The measuring unit 110 may include a camera lens 910 and an LED assisting lamp 920 of a smart phone 900. The processing unit 120 can be realized by a processing chip (not illustrated) of the smart phone 900. The storage unit 130 can be realized by a memory (not illustrated) of the smart phone 900. The user may further install specific applications (APP) for connecting the camera lens 910, the light emitting diode (LED) assisting lamp 920, the processing chip and the memory of the smart phone 900 to execute the computerized method for analyzing the physiological signal of the present embodiment of the invention.

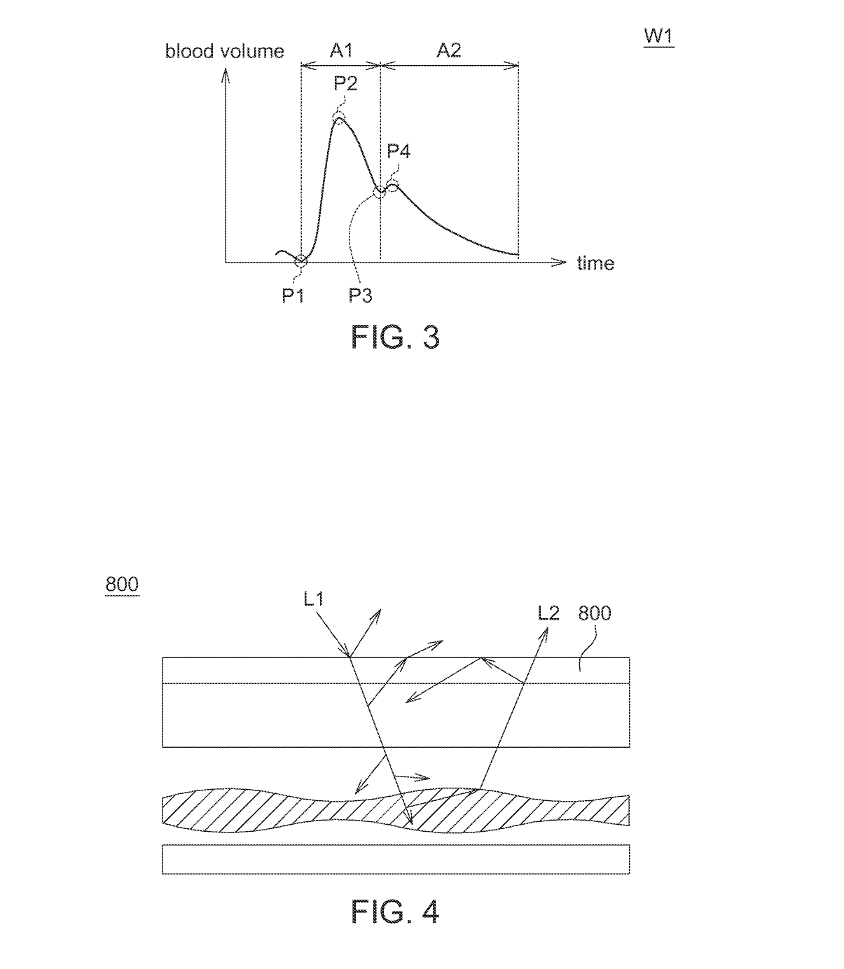

Referring to FIG. 3, a schematic diagram of a pulse waveform is shown. The measuring unit 110 measures a pulse waveform W1. The pulse waveform W1 represents a blood volume of a blood vessel over time. Significant change occurs to the blood volume inside the blood vessel between the systolic period and the diastolic period of the heart. At the pacemaker point P1, the heart enters the systolic period A1, and enters the ejection stage. At pacemaker point P1, the blood volume is at a low level. After the ejection stage, the blood volume reaches the maximum level at the percussion wave peak P2. The dicrotic notch point P3 is at the juncture between the systolic period A1 and the diastolic period A2. After the diastolic period A2, the dicrotic wave P4 reflects the change in the blood volume caused by the rebound of the extremities.

Referring to FIG. 4, a schematic diagram of the blood vessel inside a testing portion of a human body is shown. The testing portion, such as a finger 800, has denser blood capillaries and thinner tissues. After the emitted light L1 is ejected to the finger 800, the reflective light L2 is ejected to the external. After the emitted light L1 is reflected by the blood vessel, the characteristics of the emitted light L1, such as the color and the intensity of the light, change due to the influence of the blood volume of the blood vessel. The larger the blood volume of the blood vessel, the weaker the characteristics of the reflective light L2. For example, the color of the light fades.

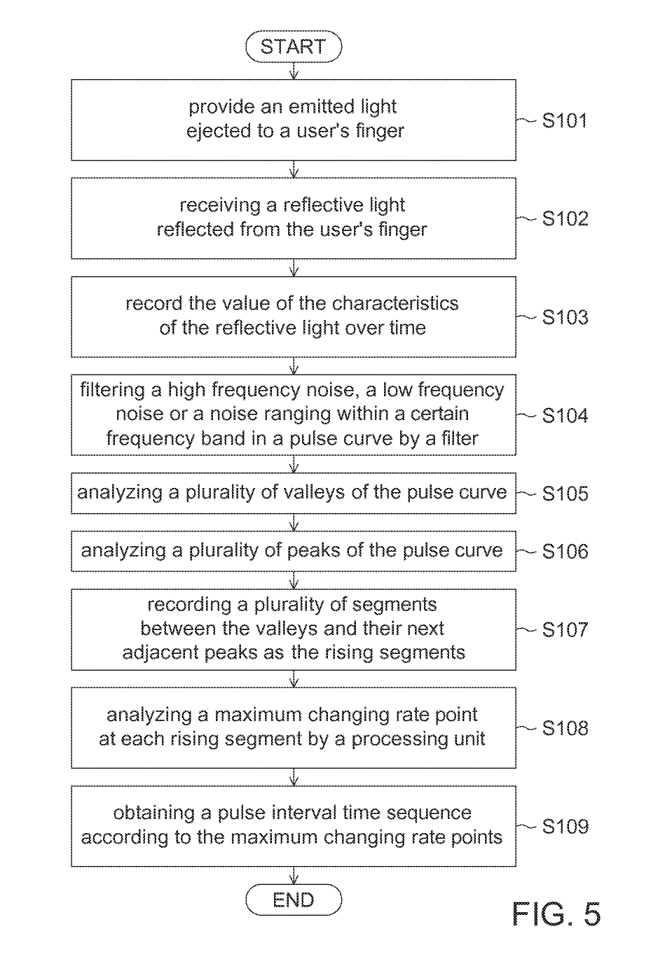

Referring to FIG. 5, a flowchart of a computerized method for analyzing a physiological signal is shown. The computerized method for analyzing the physiological signal is exemplified below with the computerized device 100 for analyzing the physiological signal of FIG. 1.

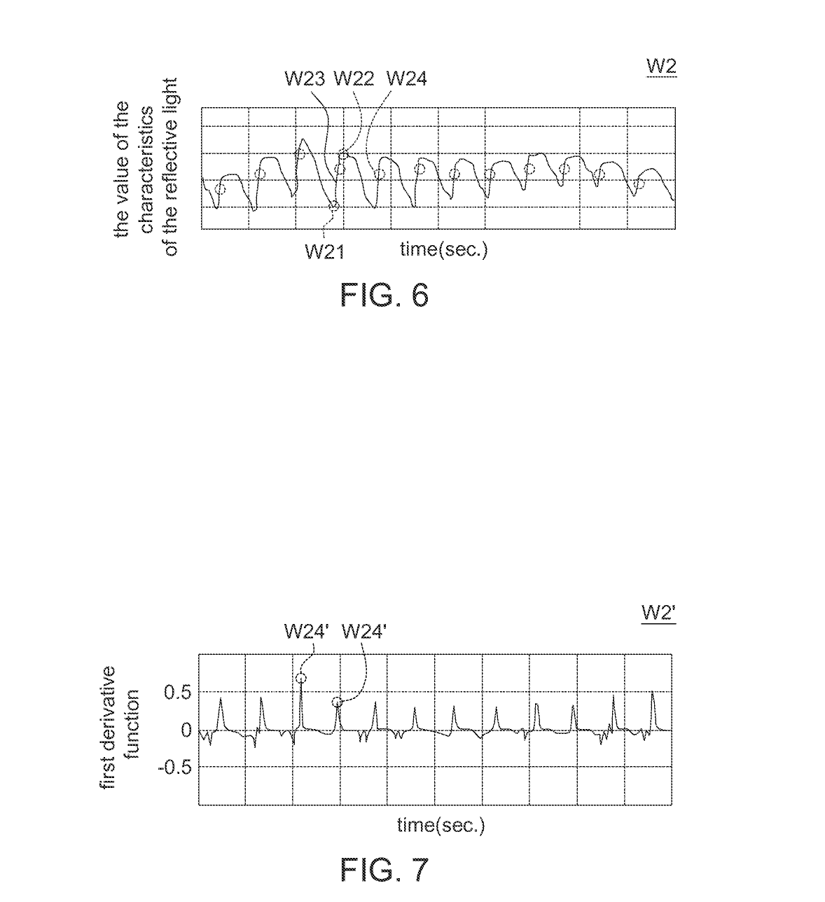

Referring to FIG. 6, a schematic diagram of a pulse waveform W2 measured by a measuring unit 110 is shown. In steps S101 to S103, a pulse waveform W2 is measured by the measuring unit 110. The pulse waveform W2 represents a blood volume of a blood vessel over time. The pulse waveform W2 can be represented in different ways. For example, the pulse waveform W2 can be realized by a curve representing a blood volume of a blood vessel over time. The pulse waveform W2 can be realized by a curve representing the characteristics of a light after passing through a blood vessel over time, such as a curve obtained by using photoplethysmography (PPG) technology. In an embodiment, the pulse waveform W2 such as represents the color or the intensity of a light over time.

As indicated in FIG. 1, the measuring unit 110 of the present embodiment of the invention includes a light emitter 111, a light receiver 112 and a sequence recorder 113. In step S101 of measuring the pulse waveform W2, an emitted light L1, such as a white light, is provided by the light emitter 111. Let FIG. 2 be taken for example. The light emitter 111 can be realized by an LED assisting lamp 920 near by the camera lens 910 of the smart phone 900. The emitted light L1 is ejected to a testing portion with denser blood capillaries and thinner tissues such as the finger 800.

In step S102, the reflective light L2 is received by the light receiver 112. Let FIG. 2 be taken for example. The light receiver 112 can be realized by such as the camera lens 910 of the smart phone 900. The camera lens 910 is adjacent to the LED assisting lamp 920. The user's finger 800 can cover the camera lens 910 and the LED assisting lamp 920 at the same time. After the reflective light L2 is reflected from the user's finger 800, the reflective light L2 is further reflected to the camera lens 910.

In step S103, the value of the characteristics of the reflective light L2 over time is recorded by the sequence recorder 113. The sequence recorder 113 can be realized by such as a chip, a firmware circuit or a computer readable recording medium storing a plurality of programming codes. In the present embodiment of the invention, the sequence recorder 113 dynamically records the red value of the reflective light L2 to generate a pulse waveform W2.

As indicated in FIG. 6, characteristics of the light of the pulse waveform W2 measured by the measuring unit 110, such as the red value of the light color, oscillate between 248 and 254. The oscillation in the red value of the pulse waveform W2 reflects the state of heat beat and pulse.

As indicated in FIG. 1, the computerized device 100 for analyzing the physiological signal of the present embodiment of the invention further includes a filter 140. In step S104, a high frequency noise, a low frequency noise or a noise ranging within a certain frequency band of the pulse waveform W2 can be further filtered off by the filter 140, so that analysis precision can be increased. In an embodiment, the computerized device 100 for analyzing the physiological signal may directly analyze the pulse waveform W2 without using the filter 140.

In steps S105 to S107, a plurality of rising segments W23 of the pulse waveform W2 are analyzed by the processing unit 120. The rising segments W23 of the pulse waveform W2 indicate that the heart is in an ejection stage.

In steps S105 to S106, as indicated in FIG. 6, a plurality of valleys W21 and a plurality of peaks W22 of the pulse waveform W2 are analyzed by the processing unit 120. Step of analyzing the valleys W21 and step of analyzing the peaks W22 can be executed concurrently or separately (the sequence of the two steps is exchangeable).

The valleys W21 and the peaks W22 are interlaced and regularly oscillate in the pulse waveform W2. In step S107, each segment between valley W21 and its next adjacent peak W22 is recorded by the processing unit 120 as a rising segment W23 to obtain a plurality of rising segments W23.

In step S108, a maximum change rate point W24 at each rising segment W23 is analyzed by the processing unit 120. Referring to FIG. 7, a schematic diagram of a first derivative function curve W2′ of the pulse waveform W2 of FIG. 6 is shown. The first derivative function curve W2′ of the pulse waveform W2 represents the change rate of the pulse curve W2. In each rising segment W23, the maximum first derivative function point W24′ is the maximum change rate point W24.

In step S109, the maximum change rate points W24 are stored to the storage unit 130. The processing unit 120 further obtains a pulse interval time sequence according to the maximum change rate points W24. The pulse interval time sequence may record the intervals between the maximum change rate points W24. The intervals are such as 0.75 second, 0.71 second and so on. Alternatively, the pulse interval time sequence may record the occurring time of each maximum change rate points W24, such as 1.66 seconds, 2.46 seconds, 3.21 seconds, 3.92 seconds, and so on. The pulse interval time sequence can be used in the analysis of the heart rate (HR), the heart rate variability (HRV) and the pulse rate variability (PRV).

In the present embodiment of the invention, the pulse interval time sequence is obtained according to the maximum change rate points W24 of the rising segments W23 of the pulse waveform W2 instead of the peaks W22 of the pulse waveform W2. The maximum change rate points W24 represent the time point at which the work is the maximum in the ejection stage. The peaks W22 of the pulse waveform W2 merely represent the maximum accumulated ejection volume in the ejection stage. The peaks W22 of the pulse waveform W2 do not occur at the time points at which the work is the maximum, and can be easily influenced by external factors such as ambient light, motion artifact, posture, and so on. In the present embodiment of the invention, the pulse interval time sequence is obtained according to the maximum change rate points W24 of the rising segments W23 of the pulse waveform W2, so that the influence of external factors are greatly reduced and analysis precision is greatly increased.

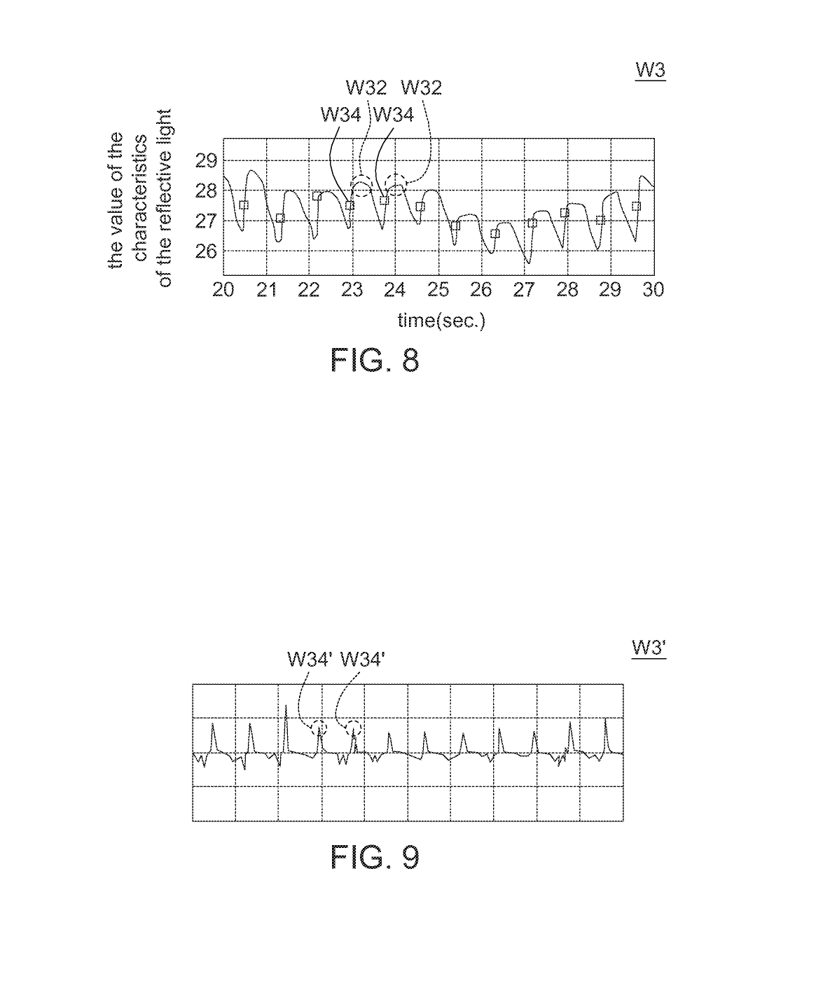

Referring to FIG. 8, a schematic diagram of another pulse waveform W3 measured by a measuring unit 110 is shown. In an embodiment, the user's finger 800 measures a pulse waveform W3 when the force is not uniformly applied. Since the force is not uniformly applied, the pulse waveform W3 is severely interfered with between 15 and 20 seconds. During this interval, since the peaks W32 do not occur at the time points at which the ejection work is the maximum, the measurement of the peaks W32 may be easily interfered with and becomes difficult.

Referring to FIG. 9, a schematic diagram of a first derivative function curve W3′ of the pulse waveform W3 of FIG. 8 is shown. FIG. 9 shows that despite the peaks W32 of the pulse waveform W3 of FIG. 8 are severely interfered with, the maximum first derivative function point W34′ still can be correctly found in FIG. 9. The maximum change rate points W34 of FIG. 8 can be obtained from the maximum first derivative function point W34′ of each first derivative function of FIG. 9.

That is, the maximum change rate points W34 has the maximum ejection work, and is thus not easily subjected to external interference. Although severe external interference occurs, the computerized method and device 100 for analyzing the physiological signal of the present embodiment of the invention still achieve high accuracy levels.

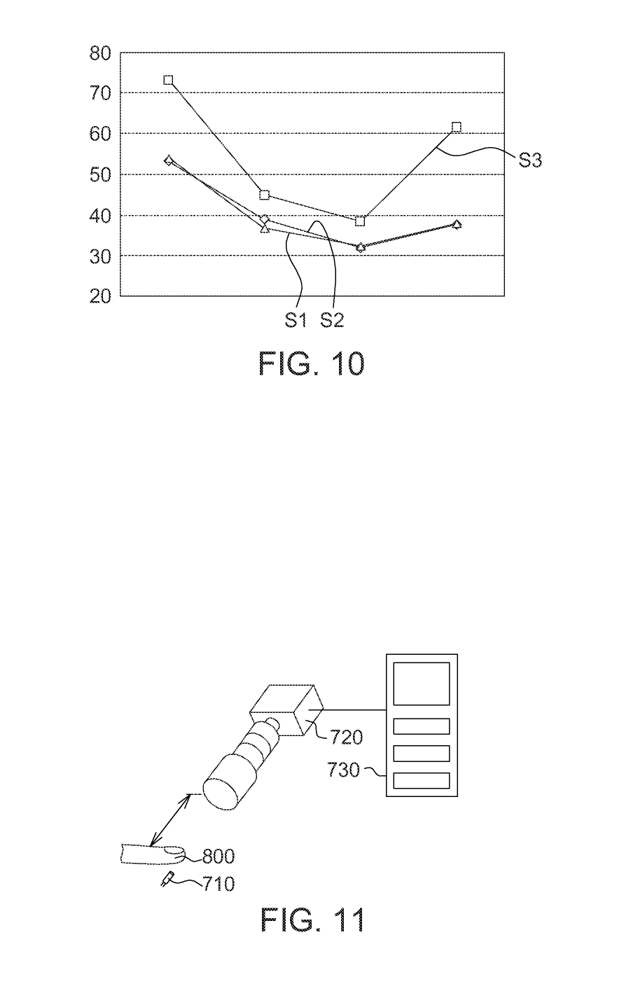

Referring to FIG. 10, a comparison of three types of HRV indexes S1, S2, S3 is shown. The first type of HRV index S1 is obtained according to the maximum change rate points W34 of the pulse waveform W3. The second type of HRV index S2 is obtained according to ECG. The third type of HRV index S3 is obtained according to the peaks W32 of the pulse waveform W3. As indicated in FIG. 10, the first type of HRV index S1 is close to the second type of HRV index S2, but the third type of HRV index S3 is deviated from the second type of HRV index S2. In general, the HRV index S2 obtained according to ECG has highest precision level. Therefore, the HRV index S1 obtained according to the maximum change rate points W34 has higher precision level.

In an embodiment, the computerized device 100 for analyzing the physiological signal can be realized by a system formed by many electronic devices. Referring to FIG. 710, a schematic diagram of light source 710, a photo-electro converter 720 and a server 730 is shown. The light emitter 111 of the measuring unit 110 can be realized by a light source 710, the light receiver 112 of the measuring unit 110 can be realized by a photo-electro converter 720, the processing unit 120 can be realized by microprocessing chip (not illustrated) and a motherboard (not illustrated) which are in-built in a server 730, and the storage unit 130 can be realized by a hard disc (not illustrated) in-built in the server 730. After the light emitted from the light source 710 passes through finger 800, the light is emitted towards the photo-electro converter 720. After the photo-electro converter 720 converts the light into an electrical signal, a pulse waveform whose vertical axis denoting electrical potential can be obtained.

The computerized method and device for analyzing physiological signal disclosed in the above embodiments can execute medical analysis in a distributed, electronized and mobilized manner, and is ideally to be taken in conjunction with a remote healthcare system and a mobile healthcare system.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A computerized method for analyzing a physiological signal, comprising:
measuring a pulse waveform by a measuring unit, wherein the pulse waveform represents a blood volume of a blood vessel over time;
analyzing a plurality of rising segments of the pulse waveform by a processing unit;
analyzing a maximum change rate point at each rising segment by the processing unit; and
obtaining a pulse interval time sequence according to the maximum change rate points.

2. The computerized method for analyzing the physiological signal according to claim 1, further comprising:
filtering a high frequency noise, a low frequency noise or a noise ranging within a certain frequency band of the pulse waveform by a filter.

3. The computerized method for analyzing the physiological signal according to claim 1, wherein the pulse waveform represents characteristics of the light after passing through the blood vessel over time.

4. The computerized method for analyzing the physiological signal according to claim 1, wherein step of measuring the pulse waveform comprises:
providing an emitted light, wherein the emitted light is ejected to a user's finger;
receiving a reflective light, wherein the reflective light is reflected from the user's finger; and
recording the value of the characteristics of the reflective light over time.

5. The computerized method for analyzing the physiological signal according to claim 1, wherein step of analyzing the rising segments of the pulse waveform comprises:
analyzing a plurality of valleys of the pulse waveform;
analyzing a plurality of peaks of the pulse waveform; and
recording a plurality of segments between the valleys and their next adjacent peaks as the rising segments.

6. The computerized method for analyzing the physiological signal according to claim 1, wherein each maximum change rate point represents the point having the maximum of the first derivative function of each rising segment.

7. A computerized device for analyzing a physiological signal, comprising:
a measuring unit used for measuring a pulse waveform, wherein the pulse waveform represents a blood volume of a blood vessel over time;
a processing unit used for analyzing a plurality of rising segments of the pulse waveform, and analyzing a maximum change rate point at each rising segment; and
a storage unit used for storing the maximum change rate points, wherein the processing unit further obtains a pulse interval time sequence according to the maximum change rate points.

8. The computerized device for analyzing the physiological signal according to claim 7, further comprising:
a filter used for filtering a high frequency noise, a low frequency noise or a noise ranging within a certain frequency band of the pulse waveform.

9. The computerized device for analyzing the physiological signal according to claim 7, wherein the pulse waveform represents characteristics of the light after passing through the blood vessel over time.

10. The computerized device for analyzing the physiological signal according to claim 7, wherein the measuring unit comprises:
a light emitter used for measuring an emitted light, wherein the emitted light is ejected to a user's finger;
a light receiver used for receiving a reflective light, wherein the reflective light is reflected from the user's finger; and
a sequence recorder used for recording the value of the characteristics of the reflective light over time.

11. The computerized device for analyzing the physiological signal according to claim 7, wherein the processing unit analyzes a plurality of valleys and a plurality of peaks of the pulse waveform, and records a plurality of segments between the valleys and their next adjacent peaks as the rising segments.

12. The computerized device for analyzing the physiological signal according to claim 7, wherein each maximum change rate point represents the point having the maximum of the first derivative function of each rising segment.
